(19) United States
(12) Patent Application Publication
(10) Pub. No.: US 2008/0226574 A1
(43) Pub. Date: Sep. 18, 2008

(54) Line of cosmetic compositions

(75) Inventor: Ludovic Thevenet, Bourg la Reine (FR)

Correspondence Address: OLIFF & BERRIDGE, PLC, P.O. BOX 320850, ALEXANDRIA, VA, 22320-4850, US

(73) Assignee: L'OREAL, PARIS (FR)

(21) Appl. No.: 11/984,187

(22) Filed: Nov. 14, 2007

Related U.S. Application Data
(60) Provisional application No. 60/876,160, filed on Dec. 21, 2006.
(60) Provisional application No. 60/876,161, filed on Dec. 21, 2006.
(60) Provisional application No. 60/899,872, filed on Feb. 7, 2007.

(30) Foreign Application Priority Data
Nov. 17, 2006 (FR) 06 54976
Nov. 17, 2006 (FR) 06 54977
Nov. 17, 2006 (FR) 06 54981
Dec. 12, 2006 (FR) 06 55453
Dec. 12, 2006 (FR) 06 55454
Dec. 12, 2006 (FR) 06 55460

(51) Int. Cl. A61K 8/26; A61K 8/25; A61K 8/28; A61K 8/29; A61K 8/18; A61K 8/02; A61Q 1/00

(52) U.S. Cl. 424/63; 424/724; 424/684; 424/691; 424/646; 424/641; 424/70.6; 424/401

(57) ABSTRACT

The present invention relates to a line of cosmetic compositions comprising at least two compositions each comprising a cosmetically-acceptable medium and at least one multilayer interference pigment, each composition providing coverage that is greater than or equal to 25, the respective multilayer interference pigments of the various compositions being selected in such a manner that the colors of the compositions in the mass, taken two by two, differ by a color difference ΔE_{beforeapplication} that is less than or equal to 3, and in such a manner that, after application, the compositions, taken two by two, present a color difference ΔE_{afterapplication} that is greater than or equal to 5.

This non provisional application claims the benefit of French Applications No^{s}. 06 54976-06 54977 and 06 54981 filed on Nov. 17, 2006 and of French Applications No^{s} 06 55453-06 55454 and 06 55460 filed on Dec. 12, 2006 and U.S. Provisional Applications Nos. 60/876,160-60/876,161 filed on Dec. 21, 2006 and U.S. Provisional Applications Nos. 60/899,872 filed on Feb. 7, 2007.

The present invention relates to cosmetic compositions, and more particularly to cosmetic compositions for making up keratinous materials.

### BACKGROUND

It is known to introduce diffusing pigments into makeup compositions so as to produce colors by a phenomenon of absorbing light using specific chromophores.

To the diffusing pigments that are necessary in order to benefit from a colored background that is continuous and sufficiently covering, it is possible to add effect particles in order to create highlights or to impart a nacre aspect, for example.

However, the intensity of the color produced by such compositions can turn out to be insufficient for obtaining a completely satisfactory result.

Multilayer interference pigments, comprising a stack of layers that have refractive indices and thicknesses that are conveniently selected in order to generate a color by an interference phenomenon, make it possible to produce a color of intensity that is greater than the color intensity of the above-mentioned diffusing pigments.

To the inventor's knowledge, in commercially-available compositions, and with the exception of powders, such multilayer interference pigments are used at a concentration by weight that does not exceed 5%.

In addition, it is known from eyeshadows of tradename CHRYSALIDE by LANCÔME to impart both coverage and color intensity by means of makeup that is applied in a first operation of depositing, on the keratinous materials, a black base coat containing a diffusing pigment that provides coverage, and then, in a second operation of depositing, on the base coat, a composition that provides color by means of a multilayer interference pigment. Without the base coat, the top coat is practically invisible since it has no covering powder and no color.

The need for two successive applications complicates applying the makeup and makes packaging it more costly.

### SUMMARY

Lines of Cosmetic Compositions

A need exists to benefit from compositions that are capable of providing coverage and of producing a saturated color, so as to make it possible, in a single operation, to obtain makeup that is both covering and colored.

In addition, it is desirable to benefit from making up keratinous materials with cosmetic compositions that present novel effects that are likely to attract consumers, without the novel effects being to the detriment of the quality of the makeup obtained.

The invention seeks in particular to satisfy all or some of the above-identified needs.

In one of its aspects, the present invention provides a line of cosmetic compositions comprising at least two compositions, each comprising a cosmetically-acceptable medium and at least one multilayer interference pigment, each composition providing coverage that is greater than or equal to 25, better 30, the respective multilayer interference pigments of the various compositions being selected in such a manner that the bulk colors of the compositions, taken two by two, differ by a color difference ΔE_{beforeapplication} that is less than or equal to 3, and in such a manner that, after application, the compositions, taken two by two, present a color difference ΔE_{afterapplication} that is greater than or equal to 5.

For the purpose of calculating ΔE, the color after application is determined after the composition has been spread on a contrast card, as when measuring coverage.

The term “line” should be understood as a plurality of compositions that are proposed to consumers, e.g. under the same trade name and in packaging.

In products offered to consumers, the line of the invention may form part of a larger line including compositions that do not correspond to the invention, e.g. proposed under the same trade name and in the same packaging as the compositions of the invention.

All of the compositions in the line may be anhydrous or they may be aqueous.

ΔE_{beforeapplication} may be less than or equal to 2,

ΔE_{afterapplication} may be greater than or equal to 5, better 6.

The coverage may be greater than 25, e.g. in the range 30 to 70, e.g. greater than any integer lying in this range, e.g. greater than or equal to 40.

Each composition may present a difference in color between the bulk color and the after-application color that is greater than or equal to 5, e.g. greater than any integer lying in the range 5 to 30.

With multilayer interference pigments, color production by the interference phenomenon is in competition with color production by the phenomenon of absorption by the surface layer of the pigment.

Thus, when the pigment concentration increases sufficiently, the color produced by the interference phenomenon decreases while that produced by absorption increases.

By exploiting this property, the invention makes it possible to observe a variation in color in each composition in the line during application, which imparts a fun aspect in use. This fun character is reinforced in that all of the compositions in the line present substantially the same bulk color.

The invention also offers novel possibilities with regard to selling cosmetic compositions, making it possible, in the packaging, to take advantage of this variation in color before and after application.

The bulk color of each composition in the line may be white. The term “white” should be understood as achromatic in the CIE meaning.

Within its bulk, each composition in the line may have a whiteness index that is greater than or equal to 40.

Each composition in the line need not include any coloring agent other than the multilayer interference pigment(s).

At least one multilayer interference pigment of the compositions in the line may comprise at least four layers, for example.

At least one multilayer interference pigment of at least one composition in the line may include a substrate made of a transparent material.

A composition in the line may include two multilayer interference pigments having layers that are made of the same material, but with at least one layer of one pigment having a thickness that is different from a corresponding layer of the other pigment, so as to produce different colors.

The compositions in the line may each include at least one multilayer interference pigment having a substrate comprising an oxide of iron or of aluminum.

The multilayer interference pigments of all of the compositions in the line may have the same dimensions to within 20%.

The multilayer interference pigments of all of the compositions in the line may have the same kind of substrate and/or the same kind of surface layer. All of the compositions in the line may have the same compounds except for the respective multilayer interference pigments. This can make it easier to obtain the same bulk color.

At least two compositions may differ from each other in only the thickness of at least one of the interference layers of their respective interference pigments.

The number of compositions in the line may lie in the range 2 to 8, for example.

The line may comprise at least three compositions having multilayer interference pigments, the total content of multilayer interference pigment(s) of the three compositions in the line respectively being less than or equal to 8%, strictly greater than 14%, and lying in the range 12% to 14%, by weight.

All of the compositions in the line may have the same multilayer interference pigment.

The line may comprise at least two, or even three or four, compositions having different respective multilayer interference pigments with the same content by weight.

All of the compositions in the line may be packaged in identical receptacles.

The invention also provides a line of cosmetic compositions that are not powders, in particular that are in liquid or stick form, each including, in a cosmetically-acceptable medium, one or more multilayer interference pigments, with, for at least one composition, a total content of multilayer interference pigment(s) that is less than or equal to 9%, and, for one composition, a content that is greater than or equal to 11%, by weight.

Each composition may be packaged with means that indicate the color of the composition after application to keratinous materials (lips, skin, nails, eyelashes, eyebrows, hair).

By way of example, the means could be a printed ink or varnish, a thin layer of composition, or a molding or overmolding of a material incorporating the multilayer interference pigment(s).

In an embodiment, the invention provides a line of cosmetic compositions comprising at least two compositions each comprising a cosmetically-acceptable medium and at least one multilayer interference pigment in a weight proportion greater than or equal to 7%, the multilayer interference pigments producing different colors after application, the multilayer interference pigments having a substrate preferentially made of a non opaque material for example made of mica, glass, silica, and preferentially comprising no colored layer, the proportion of diffusing pigment producing a color by a phenomenon of absorbing light being in each composition preferentially less than 0.5%.

### Compositions of White Color in Their Bulk

Such compositions may be used to form a line of compositions according to the invention. Such compositions may also be interesting in themselves.

In an embodiment, the invention provides a solid cosmetic composition, in particular in stick form, comprising in a cosmetically-acceptable medium at least one multilayer interference pigment suitable for conferring a white color to the composition in its bulk, and for giving rise, after application of the composition, to a color variation ΔE of at least 5.

The term “stick” is used to designate a composition in solid form, generally in the form of an elongate block, enabling makeup to be transferred by friction. By way of example, a stick can be obtained by molding or by extrusion.

For the purposes of calculating ΔE, the color after application is determined after the composition has been spread on a contrast card, as for measuring coverage.

In its bulk, the composition may have a whiteness index greater than or equal to 40.

The composition may be anhydrous or aqueous.

Depending on the composition, the difference ΔE may for example lie in the range 5 to 30, in particular it may be greater than any integer lying in that range.

The coverage of the composition may lie in the range 30 to 70, in particular it may be greater than any integer lying in said range, for example it may be greater than or equal to 40.

The amount of multilayer interference pigment may lie in the range 7% to 20% by weight, better 8% to 15%, in particular for a composition that is not in powder form, e.g. that is in stick form.

For a loose or compacted powder composition, the amount of multilayer interference pigment may lie in the range 40% to 95%, better 50% to 80%, for example.

The composition need not include any coloring agent other than the multilayer interference pigment(s).

The multilayer interference pigment may comprise at least four layers, for example.

The multilayer interference pigment may comprise a substrate of a material that is rough to a greater or lesser extent, which can enable a greater or smaller amount of gloss to be imparted to the composition.

By way of example, the substrate may be selected from relatively-rough natural mica, from synthetic mica, from alumina, from silica, or from glass or metal for a surface that is smoother.

The composition may include two multilayer interference pigments having layers made up of the same materials, at least one layer of one pigment having thickness that is different from a corresponding layer of the other pigments, so as to produce different colors.

In an embodiment, the invention provides a cosmetic composition comprising, in a cosmetically-acceptable medium, at least one multilayer interference pigment suitable for conferring a white color on the composition in its mass, of whiteness index greater than or equal to 40, the total amount of multilayer interference pigment lying in the range 7% to 20% by weight for a non-powder composition and in the range 40% to 95%, better 40% to 80% for a powder composition.

In an embodiment, the invention also provides packaging comprising:
a composition of white color in its bulk as defined above; and
means for informing the user about the color of the composition after application on keratinous materials (skin, lips, hair, nails, eyelashes, eyebrows). By way of example, that may be done by printing an ink or a varnish, by means of a thin layer of the composition, or by molding or overmolding a material that incorporates the multilayer interference pigment(s).

### Set for the Application of Two Coats

In an embodiment, the invention provides a set comprising:
a first cosmetic composition for applying to keratinous materials, comprising in a cosmetically-acceptable medium at least one multilayer interference pigment, the first composition presenting coverage greater than or equal to 25, better 30, the amount of multilayer interference pigment conferring on the composition a color change ΔE of at least 2, better 5, between its bulk color and its color after application on keratinous materials;
a second composition referred to as a top coat for applying onto the first composition; or
a second composition referred to as a base coat for applying before the first composition on the keratinous materials.

For the purpose of calculating ΔE, the color after application is determined after the composition has been spread on a contrast card, as described in detail below.

The second composition is preferably liquid.

The second composition may include a fatty phase so as to confer gloss to the makeup.

The second composition may be transparent so as to avoid affecting the saturation of the color produced by the first composition.

The second composition need have no solid bodies, so as to avoid diffusing light reflected by the first composition. In a variant, the second composition may include at least one effect pigment, in particular a metallic reflecting pigment, interference pigments, compounds having properties chosen from photochromic, thermochromic, solvatochromic, piezochromic, tribochromic, or mechanoluminescent properties.

The first composition may be anhydrous or aqueous.

Depending on the first composition, the difference ΔE may be greater than any integer value lying in the range 5 to 30.

The coverage of the first composition or of the base coat may be greater than 30, in particular greater than any integer value lying in the range 30 to 70, e.g. greater than or equal to 40.

The percentage by weight of multilayer interference pigment in the first composition may lie in the range 7% to 15%, better 8% to 15%, in particular for a first composition that is not in powder form, e.g. being liquid or in stick form.

With a first composition in powder form, the percentage of multilayer interference pigment may, for example, lie in the range 40% to 95%, better 40% to 80%.

The bulk color of the first composition may be white, i.e. it may be achromatic in the CIE meaning. The bulk color of the base coat composition may have a whiteness index greater than or equal to 40.

The first composition need not include any coloring agent other than the multilayer interference pigment(s). The first composition may include two multilayer interference pigments having layers made of the same material, at least one layer of one pigment having a thickness that is different from a corresponding layer of the other pigments so as to produce different colors.

The set may include means for showing the color of the first composition after application or the color of both compositions of the set after application to keratinous materials (skin, lips, nails, eyelashes, eyebrows, hair).

By way of example, that may be done by printing an ink or a varnish, by means of a thin layer of the composition, or a molding or overmolding of a material incorporating the multilayer interference pigment(s). The second composition as a top coat comprises a cosmetically-acceptable medium.

The formulation of this medium is selected in such a manner as to enable it to be applied on the first composition, e.g. for the purpose of conferring gloss and/or improving retention and/or shading an optical effect provided by the first composition.

The second composition as a top coat may comprise a liquid fatty body or a film-forming agent.

The second composition as a top coat may include a colorant or a plurality of active agents and other compounds.

The second composition as a top coat may present any form compatible with application onto the first composition.

When it is desired to obtain gloss, the top coat composition is preferably liquid and transparent, advantageously including a fatty phase.

The second composition may include at least one effect pigment. The pigment should be present in an amount that does not affect observing the color produced by the base coat composition once it has been applied onto keratinous materials.

The composition may be packaged in any receptacle or on any support provided for this purpose. The first and second compositions may be contained, where appropriate, in two compartments of a single packaging device and/or in a single package before first use.

Each composition may be applied by using an optionally flocked applicator, e.g. a foam, an endpiece, a paint brush, a felt, a spatula, a sintered element, a brush, a comb, or an optionally woven fabric.

Application can also be performed using a finger or by placing each composition directly on the substrate that is to be made up, for example by rubbing a stick or by spraying with the help of a piezoelectric or electrostatic device, or by transferring a layer of composition that has previously been deposited on an intermediate substrate.

The first composition can be packaged in a packaging device that makes it possible to see the bulk color of the composition.

The packaging device may be a receptacle having a body that is transparent, at least in part, and/or a packaging device including a stopper member that is transparent, at least in part.

The first composition can be packaged in a device that makes it possible to see both the bulk color of the composition and the color of the composition after application onto keratinous materials.

The first composition can be packaged in a device that includes means that are representative of the color that is revealed on application, e.g. a deposit of a layer of a composition or of an ink or a varnish that includes the same coloring agent as the first composition.

Where appropriate, the first composition or the top coat, second composition may be packaged together with a magnet, making it possible to modify the orientation of the particles of the multilayer interference pigment when said pigment presents non-zero magnetic susceptibility.

### Measuring Coverage

### Liquid Compositions (at 25° C.)

The term “liquid composition” is used to mean a composition of viscosity that can be measured. A liquid composition can flow under the effect of its own weight.

Coverage of compositions is measured at a finite thickness of 50 micrometers (μm), the liquid compositions being, for example, compositions for application to the lips, in particular liquid lipsticks, liquid lip glosses, and liquid lip balms; nail varnishes; eyeshadows; liquid foundations; mascara; and other liquid makeup that is not for application to the lips.

The composition is spread on matt-black and matt-white contrast cards, e.g. trade name Leneta Form WP1 for the matt-black card and Leneta 1A for the matt-white card.

Application can be performed with an automatic spreader.

Coverage is measured on the compositions spread in this way.

### Solid Compositions (at 25° C.)

Solid compositions are compositions of viscosity that cannot be measured.

Solid compositions are compositions cast in the form of a stick, or they are powders in free or compacted form.

a) For free- or compacted-powder solid compositions, the composition is applied using the same contrast cards as above, but that are covered in respective slightly rough transparent adhesive strips, e.g. trade name BLENDERM® by 3M and referenced 15025, which strips are bonded via their adhesive faces on the contrast cards.

The composition is deposited on the adhesive strip in such a manner as to obtain a homogenous deposit of 0.5 micrograms per square centimeter (mg/cm²)±0.02 mg/cm².

In order to perform the deposit, it is possible to use a sponge loaded with composition and mounted on a spreader appliance that causes the sponge to perform predefined movements. By way of example, the sponge is a single-use sponge of “LANCÔME-Photogenic” type, used on its pink side.

b) The stick compositions are melted, e.g. at 90° C., then spread in liquid state, with a thickness of 50 μm, onto matt-black or matt-white contrast cards, e.g. as referenced above, but not covered in BLENDERM®. The spreader bar is maintained at the same temperature as the composition, so as to avoid any thermal shock.

### Measurements and Calculations

Reflective spectra are acquired by means of a MINOLTA 3700-d spectrocolorimeter (measurement shape diffuse/8° and observation D65/10°, specular component mode excluded, small opening (CREISS)) on black and white backgrounds, contrast cards optionally being covered in BLENDERM® as indicated above.

The spectra are expressed in colorimetric co-ordinates in the CIELab76 space in the meaning of the CIE in accordance with recommendation 15:2004.

The coverage or contrast ratio is calculated by taking the arithmetic mean of Y on a black background, divided by the mean value of Y on a white background, multiplied by 100.

### Measuring the Bulk Color of the Composition

The bulk color is measured after filling a container that presents a depth that is sufficiently deep to enable the thickness of composition to be considered as being infinite for measurement purposes, e.g. a depth of 3 mm or more.

The L*, a*, and b* co-ordinates are measured with a spectrocolorimeter, e.g. of trade name MINOLTA CM-2002 (D65/10°, specular component mode excluded).

### Measuring Color After Application

The color is measured on the dark background of the contrast card, the composition being spread as described above for measuring coverage, but at a thickness of. 150 μm instead of 50 μm for liquid and non-powder compositions, in particular compositions in stick form.

The color difference ΔE is calculated as follows:

ΔE=[(a*_{bulk}−a*_{afterapplication})²+(b*_{bulk}−b*_{afterapplication})²+(L*_{bulk}−L*_{afterapplication})²]^{1/2}

When the color difference ΔE varies as a function of the observation angle because of the presence of a goniochromatic agent, the maximum difference is taken as delta E.

### Measuring Whiteness Index

After measuring color, the whiteness index is calculated at infinite thickness and in accordance with the ASTM E313-05 standard.

### Multilayer Interference Pigment

The expression “multilayer interference pigment” means a pigment that is capable of producing a color by an interference phenomenon between the light rays reflected by a plurality of superposed layers of different refractive indices, in particular a succession of layers of high and low refractive indices. The pigment may comprise a substrate, for example mica, coated with only one coating having a different refraction ratio, for example a coating of TiO2.

Any multilayer interference pigment may be envisaged.

Any color may be produced by the multilayer interference pigment, e.g. optionally being of dominant wavelength lying in the range 580 to 650 nm.

The composition may include a single multilayer interference pigment or a plurality of multilayer interference pigments having different dominant wavelengths.

The multilayer interference pigment may comprise a substrate (also known as a core) that is covered, on at least one face, by one or more layers made of materials and thicknesses that are selected such that a color is produced by interference.

Layers of the interference pigment may optionally surround the substrate which may present an optionally flat shape.

The substrate may include natural mica, synthetic mica, glass, alumina, silica, or even any metal, alloy, or metal oxide. The type of substrate could be selected as a function of the shine desired. For example, for a very shiny result, a substrate made of glass or of metal could be preferred.

The interference pigment may include more than four layers of different refractive indices.

The size of the particles of the multilayer interference pigment, given by the mean grain size at half the population, also referred to as D₅₀, lies in the range 1 μm to 2000 μm, for example, better 5 μm to 2000 μm.

The proportion of multilayer interference pigment is greater than 7%, for example, and lies in the range 7% to 20%, for example, better 8% to 15% for a non-powder, liquid, or cast composition, e.g. a stick composition, and in the range 40% to 95%, for example, better 40% to 80% for a free- or compacted-powder composition.

The coverage of the composition may essentially be a result of its multilayer interference pigment content. In a variant, at least one diffusing pigment and/or fillers may impart coverage.

Nacres are examples of suitable multilayer interference pigments.

### Nacres

The term “nacre” means colored particles of any form, which may optionally be iridescent, as produced in the shells of certain mollusks, or which are synthesized, and which exhibit a “pearlescent” coloring effect by optical interference.

Examples of nacres that may be mentioned are nacre pigments such as mica titanium coated with iron oxide, mica coated with bismuth oxychloride, mica titanium coated with chromium oxide, mica titanium coated with an organic colorant in particular, and nacre pigments based on bismuth oxychloride.

They may also be particles of mica on the surface of which at least two successive layers of metal oxides and/or organic coloring substances have been superimposed.

The nacres may have a yellow, pink, red, bronze, orangey, brown, gold, and/or coppery color or glint.

Illustrative examples of nacres suitable for being introduced as a multilayer interference pigment and that may be mentioned are gold color nacres, in particular those sold by ENGELHARD under the trade names Brillant gold 20 212G (Timica), Gold 222C (Cloisonne), Sparkle gold (Timica), Gold 4504 (Chromalite), and Monarch gold 233X (Cloisonne); bronze nacres, in particular those sold by MERCK under the trade names Bronze fine (17384) (Colorona) and Bronze (17353) (Colorona), and by ENGELHARD under the trade name Super bronze (Cloisonne); orange nacres in particular those sold by ENGELHARD under the trade names Orange 363C (Cloisonne) and Orange MCR 101 (Cosmica), and by MERCK under the trade names Passion orange (Colorona) and Matte orange (17449) (Microna); brown-tinted nacres in particular those sold by ENGELHARD under the trade names Nu-antique copper 340XB (Cloisonne) and Brown CL4509 (Chromalite); nacres with a copper glint in particular those sold by ENGELHARD under the trade name Copper 340A (Timica); nacres with a red glint, in particular those sold by MERCK under the trade name Sienna fine (17386) (Colorona); nacres with a yellow glint, in particular those sold by ENGELHARD under the trade name Yellow (4502) (Chromalite); red-tinted nacres with gold glints, in particular those sold by ENGELHARD under the trade name Sunstone G012 (Gemtone); pink nacres, in particular those sold by ENGELHARD under the trade name Tan opale G005 (Gemtone); black nacres with a gold glint, in particular those sold by ENGELHARD under the trade name Nu antique bronze 240 AB (Timica); blue nacres, in particular those sold by MERCK under the trade name Matte blue (17433) (Microna); white nacres with silvery glints, in particular those sold by MERCK under the trade name Xirona Silver; and orange-pink green-gold highlight nacres in particular those sold by MERCK under the trade names Indian summer (Xirona); and mixtures thereof.

By way of example, multilayer interference pigments presenting magnetic properties are those sold under the trade names: COLORONA BLACKSTAR BLUE, COLORONA BLACKSTAR GREEN, COLORONA BLACKSTAR GOLD, COLORONA BLACKSTAR RED, CLOISONNE NU ANTIQUE SUPER GREEN, MICRONA MATTE BLACK (17437), MICA BLACK (17260), COLORONA PATINA SILVER (17289), and COLORONA PATINA GOLD (117288) by MERCK; or indeed: FLAMENCO TWILIGHT RED, FLAMENCO 25 TWILIGHT GREEN, FLAMENCO TWILIGHT GOLD, FLAMENCO TWILIGHT BLUE, TIMICA NU ANTIQUE SILVER 110 AB, TIMICA NU ANTIQUE GOLD 212 GB, TIMICA NU-ANTIQUE COPPER 340 AB, TIMICA NU ANTIQUE BRONZE 240 AB, CLOISONNE NU ANTIQUE GREEN 828 CB, CLOISONNE NU ANTIQUE BLUE 626 CB, GEMTONE MOONSTONE G 004, CLOISONNE NU ANTIQUE RED 424, CHROMA-LITE BLACK (4498), CLOISONNE NU ANTIQUE ROUGE FLAMBE (code 440 XB), CLOISONNE NU ANTIQUE BRONZE (240 XB), CLOISONNE NU ANTIQUE GOLD (222 CB), and CLOISONNE NU ANTIQUE COPPER (340 XB) by ENGELHARD.

The multilayer interference pigment of each composition of the line may be chosen from those giving a bulk white color to the composition, for example the nacres sold by ENGELHARD under the trade names SPARKLE 110P (Timica), Flamenco blue (Flamenco), Flamenco green (Flamenco), Flamenco red (Flamenco), Flamenco violet (Flamenco), Flamenco orange (Flamenco),Silkbalnc 110W (Timica), Extra large sparkle (Timica), Flamenco sparkle Gold (Flamenco), Flamenco sparkle green (Flamenco), Flamenco sparkle orange (Flamenco), Flamenco sparkle blue (Flamenco), Flamenco sparkle violet (Flamenco), Flamenco sparkle red (Flamenco), Flamenco summit gold (Flamenco) the nacres sold by MERCK under the trade name Silk blue (Timiron), Silk green (Timiron), Silk red (Timiron), Super red (Timiron), Super green (Timiron), Super blue (Timiron), Artic Silver (Timiron),splendid copper (Timiron), Splendid Violet (Timiron); the nacres sold by ECKART under the trade name Prestige Silver (Prestige), Prestige Silver Star (Prestige), Prestige Gold (Prestige), Prestige soft gold (Prestige), Prestige silk green (Prestige), Prestige silk lilac (Prestige), Prestige silk blue (Prestige), Prestige silk red (Prestige).

The mutilayer interference pigment may be deprived of a layer of a colored material, for example chosen from FeOOH, Fe203, Cr203, TiO2-x, TiOxNy, CrPO4, KFe[Fe(CN)6], Fe3O4, TiO, TiN, FeTiO3, C, Ag, Au, Fe, Mo, Cr, W.

The multilayer interference pigment may for example only comprise on the substrate one or several layers of materials chosen from TiO2 (rutile or anatase), ZrO2, SnO2, SiO2.

The multilayer interference pigment may also be selected from the reflective interference particles.

### Reflective Interference Particles

These particles may be selected from particles of synthetic substrate at least partially coated with at least one layer of at least one metal oxide selected, for example, from oxides of titanium, in particular TiO₂, of iron, in particular Fe₂O₃, of tin, or of chromium, barium sulfate, and the following materials: MgF₂, CrF₃, ZnS, ZnSe, SiO₂, A1₂O₃, MgO, Y₂O₃, SeO₃, SiO, HfO₂, ZrO₂, CeO₂, Nb₂O₅, Ta₂O₅, MoS₂, and mixtures or alloys thereof.

Examples of such particles that may be mentioned are particles comprising a substrate of synthetic mica coated with titanium dioxide, or glass particles coated either with brown iron oxide, titanium oxide, tin oxide, or one mixture thereof such as those sold under the trade name REFLECKS® by ENGELHARD.

The multilayer interference pigment may also be a goniochromatic pigment.

### Goniochromatic Pigment

The term “goniochromatic pigment” as used in the context of the present invention means a pigment that makes it possible, when the composition is spread on a surface, to obtain a color path in the a*b* plane of the 1976 CIE color space which corresponds to a variation Dh° of the hue angle h° of at least 20° when the angle of observation is varied relative to the normal in the range 0° to 80° for light at an angle of incidence of 45°.

By way of example, the color path may be measured by means of a spectrogonioreflectometer, from INSTRUMENT SYSTEMS and referenced GON 360 GONIOMETER, after the composition has been spread in the fluid state to a thickness of 300 μm by means of an automatic spreader on a contrast card from ERICHSEN and referenced Typ 24/5, the measurements being performed on the black background of the card.

The goniochromatic pigment may, for example, be selected from multilayer interference structures and liquid crystal coloring agents.

By way of example, a multilayer structure may comprise at least two layers, each layer being produced, for example, from at least one material selected from the group constituted by the following materials: MgF₂, CeF₃, ZnS, ZnSe, Si, SiO₂, Ge, Te, Fe₂O₃, Pt, Va, Al₂O₃, MgO, Y₂O₃, S₂O₃, SiO, HfO₂, ZrO₂, CeO₂, Nb₂O₅, Ta₂O₅, TiO₂, Ag, Al, Au, Cu, Rb, Ti, Ta, W, Zn, MoS₂, cryolite, alloys, polymers, and combinations thereof.

The multilayer structure may optionally be symmetrical with respect to a central layer as regards the chemical nature of the stacked layers.

Depending on the thickness and nature of the various layers, different effects are obtained.

Examples of symmetrical multilayer interference structures are as follows: Fe₂O₃/SiO₂/Fe₂O₃/SiO₂/Fe₂O₃, a pigment having this structure being sold under the trade name SICOPEARL by BASF; MoS₂/SiO₂/mica-oxide/SiO₂/MoS₂; Fe₂O₃/SiO₂/mica-oxide/SiO₂/Fe₂O₃; TiO₂/SiO₂/TiO₂ and TiO₂/Al₂O₃/TiO₂, pigments having these structures being sold under the trade name XIRONA by MERCK (Darmstadt).

By way of example, liquid crystal coloring agents comprise silicones, or cellulose ethers onto which mesomorphic groups have been grafted. Examples of suitable liquid crystal goniochromatic particles are those sold by CHENIX, and those sold under the trade name HELICONE® HC by WACKER.

Suitable goniochromatic pigments are some nacres; pigments having effects on synthetic substrates, in particular alumina, silica, borosilicate, iron oxide, or aluminum type substrates; or interference flakes coming from a polyterephthalate film.

The material may further contain dispersed goniochromatic fibers. Such fibers could present a length that is less than 80 μm, for example.

### Cosmetically-Acceptable Medium

The term “cosmetically-acceptable medium” means a medium that is not toxic and that is suitable for application to the keratinous materials of human beings.

The cosmetically-acceptable medium is adapted to the nature of the surface on which the composition is to be applied, and also to the form in which the composition is to be packaged.

The composition of the invention may comprise an aqueous medium and/or a fatty phase that is optionally anhydrous.

### Aqueous or Fatty Phase

The composition may comprise water or a mixture of water and hydrophilic organic solvents, such as alcohols, in particular linear or branched lower monoalcohols containing 2 to 5 carbon atoms, such as ethanol, isopropanol or n-propanol, polyols such as glycerine, diglycerine, propylene glycol, sorbitol, penthylene glycol, or polyethylene glycols.

The hydrophilic phase may also contain hydrophilic C₂ ethers and C₂-C₄ aldehydes.

The water or mixture of water and hydrophilic organic solvents may be present in the composition of the invention in a content lying in the range 0 to 90%, in particular 0.1% to 90% by weight relative to the total composition weight, and preferably in the range 0 to 60% by weight, more particularly 0.1% to 60% by weight.

The composition may also include a fatty phase, in particular constituted by fat that is liquid at 25° C. and possibly fat that is solid at ambient temperature, such as waxes, pasty fats, gums, and mixtures thereof.

Fats that are liquid at ambient temperature, usually termed “oils”, that are used in the invention and that may be mentioned are amongst others: hydrocarbon-containing vegetable oils such as liquid fatty acid triglycerides containing 4 to 10 carbon atoms, for example heptanoic or octanoic acid triglycerides, or even sunflower, corn, soybean, grapeseed, sesame seed, apricot kernel, macadamia nut, castor, or avocado stone oil, caprylic/capric acid triglycerides, jojoba oil, shea nut butter oil; linear or branched hydrocarbons of mineral or synthetic origin, such as paraffin oils, in particular C₈-C₁₆ isoparaffins such as isododecane, isodecane, isohexadecane, Vaseline, polydecenes, hydrogenated polyisobutene such as Parleam®; squalane; synthesized esters and ethers, in particular fatty acids such as Purcellin oil, isopropyl myristate, 2-ethylhexyl palmitate, 2-octyldodecyl stearate, 2-octyldodecyl erucate, isostearyl isostearate; hydroxylated esters such as isostearyl lactate, octylhydroxystearate, octyldodecyl hydroxystearate, diisostearylmalate, triisocetyl citrate, fatty alcohol heptanoates, octanoates, or decanoates; polyol esters such as propylene glycol dioctanoate, neopentylglycol diheptanoate, diethyleneglycol diisononanoate; and pentaerythritol esters; fatty alcohols containing 12 to 26 carbon atoms, such as octyldodecanol, 2-butyloctanol, 2-hexyldecanol, 2-undecylpentadecanol, or oleic alcohol; fluorinated hydrocarbon and/or silicone oils; silicone oils such as volatile or non-volatile, linear or cyclic polymethyl-siloxanes (PDMS) which may be liquid or pasty at ambient temperature, such as cyclomethicones or dimethicones, optionally comprising a phenyl group, such as phenyl trimethicones, phenyltrimethylsiloxydiphenyl siloxanes, diphenylmethyldimethyl-trisiloxanes, diphenyl dimethicones, phenyl dimethicones, polymethylphenyl-siloxanes; and mixtures thereof.

The oils may be present in a content lying in the range 0.01% to 90% relative to the total composition weight.

The composition of the invention may also include one or more physiologically-acceptable organic solvents. The solvent(s), which may be lipophilic, may be present in a content lying in the range 0 to 90%, better in the range 0 to 60% by weight relative to the total composition weight, and still better in the range 0.1% to 30%.

The medium may include a liquid organic phase in which water is dispersed or emulsified.

The composition may also have a continuous fatty phase which may contain less than 5% water, in particular less than 1% water relative to its total weight, and in particular it may be in the anhydrous form.

### Film-Forming Agents

The medium may comprise a film-forming agent, in particular a film-forming polymer.

The term “film-forming agent” is used to mean an agent suitable, on its own or in the presence of an auxiliary film-forming agent, for forming a macroscopically continuous film that adheres on keratinous materials, and preferably a film that is cohesive, and better still a film presenting cohesion and mechanical properties that are such that such film can be isolated and handled in isolation, e.g. when said film is formed by casting onto a non-stick surface such as a Teflon or silicone surface.

The composition may include an aqueous phase and the film-forming polymer may be present in the aqueous phase. The film-forming polymer could be a polymer in dispersion or in solution.

The composition may include an oily phase and the film-forming polymer may be present in the oily phase. The polymer could thus be in dispersion or in solution.

Suitable film-forming polymers that may be mentioned include synthetic polymers, of the radical or polycondensate type, natural polymers, and mixtures thereof.

Radical type film-forming polymers may in particular be vinyl polymers or copolymers, in particular acrylic polymers.

Examples of film-forming polycondensates that can be mentioned include polyurethanes, polyesters, polyester amides, polyamides, epoxyester resins, and polyureas.

Polyesters may be obtained in known manner by polycondensation of dicarboxylic acids with polyols, in particular diols.

Polyester amides may be obtained in similar manner to polyesters, by polycondensation of diacids with diamines or amino alcohols.

By way of example of a liposoluble film-forming polymer, mention can be made of copolymers of vinyl ester (the vinyl group being directly connected to the oxygen atom of the ester group and the vinyl ester having a saturated, linear, or branched hydrocarbon radical with one to 19 carbon atoms bonded to the carbonyl of the ester group) and at least one other monomer which may be: a vinyl ester (different from the already-present vinyl ester); an α-olefin having eight to 28 carbon atoms); an alkyl vinyl ether (in which the alkyl group has two to 18 carbon atoms); or an allyl or methallyl ester (having a saturated, linear, or branched hydrocarbon radical with one to 19 carbon atoms bonded to the carbonyl of the ester group).

These copolymers may be cross-linked with the help of agents that may be either of the vinyl type or else of the allyl or methallyl type, such as: tetraallyloxyethane; divinyl benzene; divinyl octane dioate; divinyl dodecane dioate; and divinyl octadecane dioate.

As examples of these copolymers, mention can be made of the following copolymers: vinyl acetate and allyl stearate; vinyl acetate and vinyl laurate; vinyl acetate and vinyl stearate; vinyl acetate and octadecene; vinyl acetate and octadecyl vinyl ether; vinyl propionate and allyl laurate; vinyl propionate and vinyl laurate; vinyl stearate and 1-octadecene; vinyl acetate and 1-dodecene; vinyl stearate and ethyl vinyl ether; vinyl propionate and cetyl vinyl ether; vinyl stearate and allyl acetate; vinyl dimethyl-2,2 octanoate and vinyl laurate; allyl dimethyl-2,2 pentanoate and vinyl laurate; vinyl dimethyl propionate and vinyl stearate; allyl dimethyl propionate and vinyl stearate; vinyl propionate and vinyl stearate, cross-linked with 0.2% divinyl benzene; vinyl dimethyl propionate and vinyl laurate cross-linked with 0.2% divinyl benzene; vinyl acetate and octadecyl vinyl ether, cross-linked with 0.2% tetraallyl oxyethane; vinyl acetate and allyl stearate, cross-linked with 0.2% divinyl benzene; vinyl acetate and 1-octadecene, cross-linked with 0.2% divinyl benzene; and allyl propionate and allyl stearate cross-linked with 0.2% divinyl benzene.

The film-forming polymer may also be selected from silicone resins that are generally soluble or swellable in silicone oils, constituted by cross-linked polyorganosiloxane polymers.

The film-forming polymer may also be present in the composition in the form of particles in dispersion in an aqueous phase or in a non-aqueous solvent phase, generally known as a latex or a pseudolatex. Techniques for preparing such dispersions are well known to the person skilled in the art.

The composition of the invention may comprise a plasticizer agent which encourages the formation of a film with the film-forming polymer. Such a plasticizer agent may be selected from any compound that is known to the person skilled in the art as being suitable for fulfilling the desired function.

Naturally, this list of polymers is not exhaustive.

### Other Coloring Agents

The composition can include one or more diffusing pigments, generating a color by an absorption phenomenon, in a proportion that makes it possible to preserve the interference phenomenon that is responsible for the color of the composition once applied to keratinous materials. The composition may not comprise other coloring agents than the multilayer interference pigment(s), for example not comprise pigments comprising iron oxides or other pigments producing colors by a phenomenon of absorbing light.

Each composition of the line may for example comprise less than 0.5% by weight of pigments producing a color by a phenomenon of absorbing light, for example not more than 0.5% of pigments having iron oxides, for example not more than 0.2%.

When diffusing pigments are present, various diffusing pigments can be envisaged, being selected, for example, from organic lakes or pigments that are selected in particular from the following materials and mixtures thereof:
cochineal carmine;
the organic pigments of azo, anthraquinone, indigo, xanthene, pyrene, quinoline, triphenylmethane, or fluorane dyes; and
organic lakes or insoluble salts of sodium, potassium, calcium, barium, aluminum, zirconium, strontium, titanium, or of acid dyes such as azo, anthraquinone, indigo, xanthene, pyrene, quinoline, triphenylmethane, or fluorine dyes, which dyes may comprise at least one carboxylic or sulfonic acid group.

Organic pigments that may be mentioned include those with the following denominations: D&C Blue No.4, D&C Brown No.1, D&C Green No.5, D&C Green No.6, D&C Orange No.4, D&C Orange No.5, D&C Orange N10, D&C Orange No.11, D&C Red No.6, D&C Red No.7, D&C Red No.17, D&C Red No.21, D&C Red No.22, D&C Red No.27, D&C Red No.28, D&C Red No.30, D&C Red N31, D&C Red No.33, D&C Red No.34, D&C Red No.36, D&C Violet No.2, D&C Yellow No.7, D&C Yellow No.8, D&C Yellow No.10, D&C Yellow No.11, FD&C Blue No.1, FD&C Green No.3, FD&C Red No.40, FD&C Yellow No.5, FD&C Yellow No.6.

The lake may be supported by an organic support such as colophane or aluminum benzoate, for example.

Particular organic lakes that may be mentioned include those with the following denominations: D&C Red No.2 Aluminum lake, D&C Red No.3 Aluminum lake, D&C Red No.4 Aluminum lake, D&C Red No.6 Aluminum lake, D&C Red No.6 Barium lake, D&C Red No.6 Barium/Strontium lake, D&C Red No.6 Strontium lake, D&C Red No.6 Potassium lake, D&C Red No.7 Aluminum lake, D&C Red No.7 Barium lake, D&C Red No.7 Calcium lake, D&C Red No.7 Calcium/Strontium lake, D&C Red No.7 Zirconium lake, D&C Red No.8 Sodium lake, D&C Red No.9 Aluminum lake, D&C Red No.9 Barium lake, D&C Red No.9 Barium/Strontium lake, D&C Red No.9 Zirconium lake, D&C Red No.10 Sodium lake, D&C Red No.19 Aluminum lake, D&C Red No.19 Barium lake, D&C Red No.19 Zirconium lake, D&C Red No.21 Aluminum lake, D&C Red No.21 Zirconium lake, D&C Red No.22 Aluminum lake, D&C Red No.27 Aluminum lake, D&C Red No.27 Aluminum/Titanium/Zirconium lake, D&C Red No.27 Barium lake, D&C Red No.27 Calcium lake, D&C Red No.27 Zirconium lake, D&C Red No.28 Aluminum lake, D&C Red No.30 lake, D&C Red No.31 Calcium lake, D&C Red No.33 Aluminum lake, D&C Red No.34 Calcium lake, D&C Red No.36 lake, D&C Red No.40Aluminum lake, D&C Blue No.1 Aluminum lake, D&C Green No.3 Aluminum lake, D&C Orange No.4 Aluminum lake, D&C Orange No.5 Aluminum lake, D&C Orange No.5 Zirconium lake, D&C Orange No.10 Aluminum lake, D&C Orange No.17 Barium lake, D&C Yellow No.5 Aluminum lake, D&C Yellow No.5 Zirconium lake, D&C Yellow No.6 Aluminum lake, D&C Yellow No.7 Zirconium lake, D&C Yellow No.10 Aluminum lake, FD&C Blue No.1Aluminum lake, FD&C Red No.4 Aluminum lake, FD&C Red No.40Aluminum lake, FD&C Yellow No.5 Aluminum lake, FD&C Yellow No.6 Aluminum lake.

The chemical materials corresponding to each of the organic coloring substances listed above are mentioned in the work entitled “International Cosmetic Ingredient Dictionary and Handbook”, 1997 edition, pages 371 to 386 and 524 to 528, published by “The Cosmetic, Toiletry, and Fragrance Association”, the contents of which are incorporated herein by reference.

The diffusing pigment can be a composite pigment, comprising a core that is coated at least in part by a membrane. In particular, such a composite pigment may be composed of particles comprising an inorganic core and at least one at least partial coating of at least one organic coloring substance. At least one binder may advantageously contribute to fixing the organic coloring substance onto the inorganic core.

The particles of composite pigment may have a variety of forms. In particular, said particles may be in the form of flakes or they may be globular, in particular spherical, and may be hollow or solid. The term “in the form of flakes” means particles for which the ratio of the largest dimension to the thickness is 5 or more. A composite pigment may, for example, have a specific surface area in the range 1 square meter per gram (m²/g) to 1000 m²/g, in particular in the range about 10 m²/g to about 600 m²/g, and in particular in the range about 20 m²/g to about 400 m²/g. The specific surface area is the value measured using the BET (Brunauer-Emmett-Teller) method. The proportion by weight of the core can exceed 50% relative to the total weight of the composite pigment, e.g. lying in the range 50% to 70%, e.g. 60% to 70%.

The coloring agent can also be a colorant.

The colorant can be of plant, animal, or inorganic origin, in particular of plant or inorganic origin, in particular of plant origin. The colorant can be of non-synthetic type.

The colorant can be a natural hydrosoluble or liposoluble colorant.

By way of illustration, suitable natural hydrosoluble coloring agents that may be mentioned in particular for use in the invention are: caramel; beetroot juice; carmine; betanine (beetroot); copper chlorophyllin; methylene blue; anthocyanin (enocianin, black carrot, hibiscus, elder); and riboflavin.

By way of illustration, suitable natural liposoluble coloring agents that may be mentioned in particular for use in the invention are: Sudan red; β-carotene; carotenoids; lycopene; palm oil; Sudan brown; quinoline yellow; xanthophylls (capsanthin, capsorubin, lutein); and curcumin.

Other suitable natural colorants that may be mentioned more particularly are: anthcyanin flowers or fruit or derivatives thereof; flavonoids and tannin extracts of native or fermented plants; juglone; lawsone; extracts of fermented soya, algae, mushrooms, micro-organisms; Flavylium salts non-substituted in position 3 as described in patent EP 1 172 091; extracts of Gesneria Fulgens, Blechum Procerum, Saxifraga; and pigments that can be extracted with an organic or hydro-organic solvent from a culture medium of a microfungus of the monascus Monascus type.

Suitable synthetic colorants that may be mentioned are synthetic liposoluble colorants such as DC Red 17, DC Red 21, DC Red 27, DC Green 6, DC Yellow 11, DC Violet 2, and DC Orange 5, for example. Suitable synthetic hydrosoluble colorants that may be mentioned are FDC Red 4, DC Red 6, DC Red 22, DC Red 28, DC Red 30, DC Red 33, DC Orange 4, DC Yellow 5, DC Yellow 6, DC Yellow 8, FDC Green 3, DC Green 5, and FDC Blue 1.

### Fillers

The cosmetic composition may include fillers.

The term “fillers” means particles of any form which are insoluble in the composition medium regardless of the temperature at which the composition is manufactured. Said fillers primarily act to modify the rheology or texture of the composition.

Examples of fillers that may be mentioned include talc, mica, silica, kaolin, and powders of polyamide (Nylon® or Orgasol® by Atochem).

The content in fillers is selected in such a manner as not to impede unduly the desired result.

### Active Ingredients and Other Compounds

The cosmetic composition may also contain one or more cosmetically, dermatologically, hygienically, or pharmaceutically active ingredients.

Suitable cosmetically, dermatologically, hygienically, or pharmaceutically active ingredients for use in the compositions of the invention that may be mentioned are moisturizing agents (polyols such as glycerine), vitamins (C, A, E, F, B, or PP), essential fatty acids, essential oils, ceramides, sphingolipids, liposoluble or nanoparticle sun screens, and specific skin treatment active ingredients (protective agents, antibacterials, anti-wrinkle agents, etc). Said active ingredients may be used in concentrations in the range 0.001% to 15%, for example, relative to the total weight of the composition.

The cosmetic composition may also contain ingredients which are routinely used in cosmetics, such as thickeners, surfactants, oligo-elements, moisturizing agents, softeners, sequestrating agents, fragrances, alkalinizing or acidifying agents, preservatives, antioxidants, UV filters, or mixtures thereof.

Depending on the envisaged application, the composition may also include constituents which are conventionally used in the fields under consideration, and which are present in quantities appropriate to the desired dosage or “galenical” form.

### Forms

Each cosmetic composition in the line may be in any form that is normally used for topical application, in particular in the anhydrous or non-anhydrous form, in the solid or semi-solid form of an oily or aqueous solution, an oily or aqueous gel, an oil-in-water emulsion, a water-in-oil emulsion, a multiple emulsion, a dispersion of oil in water due to vesicles located on the oil/water interface, or a spray.

All of the compositions in the line preferably present the same form.

The term “anhydrous composition” means a composition having less than 5% by weight of water, better less than 3%, or even less than 1%. An anhydrous composition need not include water that has been intentionally added during the preparation of the composition.

Each composition in the line may in particular be in the form of a stick.

Each of the cosmetic compositions in the line may constitute amongst others kinds of makeup: lipstick; liquid gloss; lipstick paste; blusher; eye-contour composition; eyeliner; mascara; nail varnish; eyeshadow; foundation; or body or hair makeup.

All of the compositions in the line may be for making up the skin, the lips, the nails, the eyelashes or the eyebrows, or the hair.

All of the compositions in the line may be obtained in accordance with the methods of preparation conventionally used in cosmetics.

### Packaging and Methods of Application

Each composition in the line may be packaged in any receptacle or on any support provided for this purpose.

Each composition can be applied using an optionally-flocked applicator, e.g. a foam, an endpiece, a paint brush, a felt, a spatula, a sintered element, a brush, a comb, or an optionally-woven fabric.

Application can also be performed using a finger or by placing the composition directly on the surface that is to be made up, for example by rubbing a stick or by spraying with the help of a piezoelectric device, or by transferring a layer of composition that has previously been deposited on an intermediate surface.

Where appropriate, each composition can be applied as a base coat that is covered by a top coat for imparting shine, for example, or applied as a top coat on a base coat, or even applied between a base coat and a top coat.

Each composition can be packaged in a packaging device that makes it possible to see the bulk color of the composition.

The packaging device is a receptacle having a body that is transparent, at least in part, and/or includes a stopper member that is transparent, at least in part.

Each composition can be packaged in a device that makes it possible to see both the bulk color of the composition and the color of the composition after application to keratinous materials.

Each composition can also be packaged in a device that includes means that are representative of the color that is revealed on application, e.g. a deposit of a layer of the composition or of an ink or a varnish that includes the same coloring agents as the composition.

Where appropriate, each composition can be packaged together with a magnet, making it possible to modify the orientation of the particles of the multilayer interference pigment, when the pigment presents non-zero magnetic susceptibility.

### Presentation Method and Display Stand

In another of its aspects, the invention also provides a method of presenting a line of cosmetic compositions in accordance with the invention, the method comprising the steps consisting in:
illuminating at least a first region of a sample in the line with diffused lighting so as to make its bulk color appear; and
illuminating at least a second region of a sample in the line with directional lighting so as to make its interference color appear.

The first and second regions can be different regions of the same sample of composition.

In a variant, the first and second regions belong to different samples.

Still in a variant, the first and second regions correspond to the same region, and the diffused and directional lighting are applied in alternation.

The invention also provides a presentation method in which:
two compositions in the line are illuminated in such a manner as to cause the same bulk color to appear; and
the two compositions are illuminated in such a manner as to reveal different colors after application, since the multilayer interference pigments of the compositions are different.

In another of its aspects, the invention also provides a display stand comprising:
a directional-lighting source;
a diffused-lighting source; and
a support that makes it possible to expose at least one sample in the line, better two samples coming from different compositions in the line, to the diffused-lighting source, and at least one sample, better both samples, to the directional-lighting source.

### Method of Applying Makeup

The invention also provides a method of making up keratinous material using a composition in a line of the invention.

The method can be for making up the skin, the lips, the nails, the eyelashes, the eyebrows, or the hair.

### Kit

The present invention also provides a makeup kit comprising:
a first composition in a line of the invention; and
a second composition comprising a cosmetically-acceptable medium for applying under or on top of the first composition.

By way of example, the second composition is for improving the durability of the first composition and/or for modifying the appearance thereof.

### PROPOSED EXAMPLES

### Example 1

### Line of Four Lipsticks

| | |
|---|---|
| Octyl-2 dodecanol | 10 |
| Ditertiobutyl 4-hydroxytoluene | 0.07 |
| Polybutene (monoolefins/isoparaffins 95/5) | 37 |
| (PM: 2060) | |
| A mixture of isopropyl, isobutyl, and n-butyl | 0.6 |
| p-hydroxybenzoates (40/30/30) | |
| Pentaerythrityl tetraisostearate | 11.33 |
| Tridecyl trimellitate | 11 |
| 2-decyl tetradecanoic acid triglyceride | 15 |
| (GUERBET C24) | |
| Multilayer interference pigment Pₖ* | 15 |

*P₁: XIRONA RED by MERCK for the first composition in the line. P₂: XIRONA BLUE by MERCK for the second composition in the line. P₃: XIRONA GREEN by MERCK for the third composition in the line. P₄: XIRONA SILVER by MERCK for the fourth composition in the line.

### Example 2

### Line of Three Lipsticks

| | |
|---|---|
| Tridecyl trimellitate | 11 |
| Liquid lanolin | Yₖ |
| Isostearyl malate | 13 |
| Acetylated lanolin | 10 |
| Lauric/palmitic/cetylic/stearic acid triglycerides | 5 |
| (50/20/10/10) | |
| Microcrystalline wax (C20-C60) | 3 |
| Protected isopropyl lanolate | 10 |
| Octyl-2-decanol | Zₖ |
| Phenyl trimethylsiloxytrisiloxane | 4 |
| (VISCOSITY: 20 CST - PM: 372 | |
| Polyethylene wax (PM: 500) | 8 |
| Multilayer interference pigment* | Xₖ |

*TIMIRON SILK RED by MERCK.
The contents Xₖ are X₁ = 13, X₂ = 7.2, and X₃ = 14.9 respectively for the three compositions in the line.
The contents Yₖ are Y₁ = 9, Y₂ = 10.5, and Y₃ = 8.5 respectively for the three compositions in the line.
The contents Zₖ are Z₁ = 14, Z₂ = 18.3, and Z₃ = 12.6 respectively for the three compositions in the line.

The following coverages are obtained:

Composition 1 (X₁=13) coverage: 41% Composition 2 (X₂=7.2) coverage: 32% Composition 3 (X₃=14.9) coverage: 42.60%

### Example 3

### Line of Three Liquid Foundations

| | |
|---|---|
| Triethanolamine | 1 |
| Disodium ethylene diamino tetraacetic acid salt, | 0.2 |
| 2H₂0 | |
| Cross-linked carboxyvinyl homopolymer | 0.5 |
| Polyvinylpyrrolidone | 0.6 |
| Glycerol | 5.75 |
| Demineralized water | 80.45 |
| 1,3-butylene glycol 1,3 | 2 |
| Silica microsphere (3 μm) | 1.5 |
| Multilayer interference pigment Pₖ* | 8 |

*P₁: Sicopearl fantastio ROSE X by BASF for the first composition. P₂: Sicopearl fantastio RUBIS X by BASF for the second composition. P₃: Sicopearl fantastio VERT X by BASF for the third composition.

### Example 4

### Line of Four Water-Based Nail Varnishes

| | |
|---|---|
| Tetrasodium pyrophosphate | 0.2 |
| Methoxy-terminated oxyethylene | 0.5 |
| polydimethylsiloxane | |
| A mixture of aliphatic polyurethane, N-methyl | 68 |
| pyrrolidone, triethylamine, and water | |
| (35/8.5/2/54.5) | |
| Glycerol | 1 |
| Demineralized water | 15 |
| Ethyl alcohol (96°) | 2.8 |
| Synthetic laponite (mixed magnesium/lithium/sodium | 1.3 |
| silicate) | |
| Multilayer interference pigment Pₖ* | 11.2 |

*P₁: XIRONA RED by MERCK for the first composition. P₂: XIRONA BLUE by MERCK for the second composition. P₃: XIRONA GREEN by MERCK for the third composition. P₄: XIRONA SILVER by MERCK for the fourth composition in the line.

### Example 5

### Line of Four Organic-Solvent Based Nail Varnishes

| | |
|---|---|
| Nitrocellulose | 11 |
| N-ethyl o,p-toluenesulfonamide | 5 |
| Alkycle resin | 10 |
| Isopropanol | 4 |
| Multilayer interference pigment Pₖ* | 10 |
| Butyl acetate/ethyl acetate 50/50 | Qsp |
| | 100 |

*P₁: XIRONA RED by MERCK for the first composition. P₂: XIRONA BLUE by MERCK for the second composition. P₃: XIRONA GREEN by MERCK for the third composition. P₄: XIRONA SILVER by MERCK for the fourth composition in the line.

### Example 6

### Line of Four Powder Eyeshadows

| | |
|---|---|
| TERTIOBUTYL 4-HYDROXYANISOLE | 0.012 |
| DITERTIOBUTYL 4-HYDROXYTOLUENE | 0.012 |
| WHITE VASELINE | 1.2 |
| OLEIC ALCOHOL | 1.2 |
| PROTECTED LIQUID LANOLIN | 0.66 |
| VASELINE OIL | 6.516 |
| CASTOR OIL | 1.296 |
| ISOPROPYL MYRISTATE | 0.864 |
| PROPYL P-HYDROXYBENZOATE | 0.24 |
| A MIXTURE OF METHYL, ETHYL, PROPYL, BUTYL, | 0.6 |
| ISOBUTYL P-HYDROXY-BENZOATES AND PHENOXY-2 | |
| ETHANOL | |
| Multilayer interference pigment Pₖ* | 50.3 |
| MAGNESIUM STEARATE | 4 |
| TALC | 30.1 |
| Metallic-reflection pigment** | 3 |

*P₁: XIRONA RED by MERCK for the first composition. P₂: XIRONA BLUE by MERCK for the second composition. P₃: XIRONA GREEN by MERCK for the third composition. P₄: XIRONA SILVER by MERCK for the fourth composition in the line.
**METASHINE ME 2040 PS by NIPPON SHEET GLASS.

### Example 7

### Line of Four Liquid Eyeshadows

| | |
|---|---|
| DITERTIOBUTYL 4-HYDROXYTOLUENE | 0.09 |
| DISTEARYLDIMETHYLAMMONIUM MODIFIED | 3.74 |
| HECTORITE | |
| LAURIC/PALMITIC/CETYLIC/STEARIC ACID | 6.46 |
| TRIGLYCERIDES (50/20/10/10) | |
| PROPYLENE CARBONATE | 1.22 |
| WHITE BEESWAX. | 7.77 |
| PROTECTED SHOREA BUTTER | 1.7 |
| PROTECTED LIQUID FRACTION OF SHEA NUT BUTTER | 0.85 |
| NYLON-12 POWDER | 10.4 |
| ISODODECANE | 35.32 |
| PROPYL P-HYDROXYBENZOATE | 0.17 |
| PROTECTED REFINED PARAFFIN | 3.88 |
| TALC | 10.4 |
| Multilayer interference pigment Pₖ* | 15 |
| Metallic-reflection pigment** | 3 |

*P₁: XIRONA RED by MERCK for the first composition. P₂: XIRONA BLUE by MERCK for the second composition. P₃: XIRONA GREEN by MERCK for the third composition. P₄: XIRONA SILVER by MERCK for the fourth composition in the line.
**METASHINE ME 2040 PS by NIPPON SHEET GLASS.

Each composition of the line may be associated to a top coat or a base coat. Such compositions are described hereinafter.

### Example 8

### Lipstick

### First Composition

| | |
|---|---|
| Tridecyl trimellitate | 11 |
| Liquid lanolin | 10 |
| Isostearyl malate | 13 |
| Acetylated lanolin | 10 |
| Lauric/palmitic/cetylic/stearic acid | 5 |
| triglycerides (50/20/10/10) | |
| Microcrystalline wax (C20-C60) | 3 |
| Protected isopropyl lanolate | 10 |
| Octyl-2-decanol | 16 |
| Phenyl trimethylsiloxytrisiloxane | 4 |
| (VISCOSITY: 20 CST - PM: 372) | |
| Polyethylene wax (PM: 500) | 8 |
| Multilayer interference pigment* | 10 |

*TIMIRON SILK RED by MERCK.

### Second Composition (Top Coat)

| | |
|---|---|
| Octyl-2 dodecanol | 10 |
| DITERTIOBUTYL 4-hydroxytoluene | 0.07 |
| Polybutene (monoolefins/isoparaffins 95/5) | 50 |
| (PM: 2060) | |
| A mixture of isopropyl, isobutyl, and n-butyl | 0.4 |
| p-hydroxybenzoates (40/30/30) | |
| Pentaerythrityl tetraisostearate | 11.33 |
| Tridecyl trimellitate | 12 |
| 2-decyl tetradecanoic acid triglyceride | 11 |
| (GUERBET C24) | |
| Effect pigment** | 3 |

**METASHINE ME 2040 PS by NIPPON SHEET GLASS.

The first composition makes it possible to generate a uniform layer of makeup having a red color that is very intense. The second composition serves to create a magnifying glass effect accompanied by a silver highlight that gives relief to the overall result of the makeup.

### Example 9

### Lipstick

### Base Coat

| | |
|---|---|
| Tridecyl trimellitate | 12 |
| Liquid lanolin | 11 |
| Isostearyl malate | 14 |
| Acetylated lanolin | 11 |
| Lauric/palmitic/cetylic/stearic acid | 6 |
| triglycerides (50/20/10/10) | |
| Microcrystalline wax (C20-C60) | 3 |
| Protected isopropyl lanolate | 10 |
| Octyl-2-decanol | 16 |
| Phenyl trimethylsiloxytrisiloxane | 4 |
| (VISCOSITY: 20 CST - PM: 372) | |
| Polyethylene wax (PM: 500) | 8 |
| Black iron oxide (Fe₃O₄) pigment* | 5 |

*TIMIRON SILK RED by MERCK.

### Composition Containing Multilayer Interference Pigment

| | |
|---|---|
| Octyl-2 dodecanol | 10 |
| DITERTIOBUTYL 4-hydroxytoluene | 0.07 |
| Polybutene (monoolefins/isoparaffins 95/5) | 45.2 |
| (PM: 2060) | |
| A mixture of isopropyl, isobutyl, and n-butyl | 0.4 |
| p-hydroxybenzoates (40/30/30) | |
| Pentaerythrityl tetraisostearate | 11.33 |
| Tridecyl trimellitate | 12 |
| 2-decyl tetradecanoic acid triglyceride | 11 |
| (GUERBET C24) | |
| Multilayer interference pigment** | 10 |

**TIMIRON SILK RED by MERCK.

The base coat is applied first and serves to generate a uniform layer of makeup having a very intense black color. The composition containing the multilayer interference pigment is applied thereon and serves to create a color effect giving a very intense makeup result.

Naturally, the invention is not limited to the examples given above. The term “comprising a” is synonymous with the term “comprising at least one”, and “lying in the range” should be understood as including the limits of the range.

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A line of cosmetic compositions comprising at least two compositions each comprising a cosmetically-acceptable medium and at least one multilayer interference pigment, each composition providing coverage that is greater than or equal to 25, the respective multilayer interference pigments of the various compositions being selected in such a manner that the colors of the compositions in the mass, taken two by two, differ by a color difference ΔE_{beforeapplication} that is less than or equal to 3, and in such a manner that, after application, the compositions, taken two by two, present a color difference ΔE_{afterapplication} that is greater than or equal to 5.

2. A line according to claim 1, with ΔE_{beforeapplication}≦2.

3. A line according to claim 1, each of the compositions in the line including at least one multilayer interference pigment having a substrate made of glass, silica, mica, alumina, or metal.

4. A line according to claim 1, the compositions in the line each including at least one multilayer interference pigment having a substrate comprising an oxide of iron or of aluminum.

5. A line according to claim 1, the multilayer interference pigments of the compositions in the line having the same kind of substrate.

6. A line according to claim 1, all of the compositions in the line having the same compounds except for the respective multilayer interference pigments.

7. A line according to claim 1, all of the compositions in the line presenting a bulk color that is substantially white.

8. A line according to claim 1, the compositions in the line not including any coloring agent other than the multilayer interference pigments.

9. A line according to claim 1, the compositions each comprising less than 0.5% by weight of pigments generating a color by a phenomenon of absorbing light.

10. A line according to claim 9, the compositions each comprising less than 0.5% by weight of pigments comprising iron oxide.

11. A line according to claim 1, the multilayer interference pigment being deprived of a colored layer.

12. A line according to claim 1, the multilayer pigment only comprising on the substrate one or several layers chosen from TiO2, ZrO2, SnO2, SiO2.

13. A line according to claim 1, the multilayer interference pigment being deprived of Fe2O3.

14. A line according to claim 1, the weight proportion of the multilayer interference pigment being greater than 7%.

15. A line according to claim 1, at least two compositions in the line including multilayer interference pigments that differ from each other in only the thickness of at least one of the interference layers.

16. A line according to claim 1, at least two compositions in the line including multilayer interference pigments having the same kind of surface layer.

17. A line according to claim 1, comprising at least three compositions having multilayer interference pigments, the total content of multilayer interference pigment(s) of the three compositions in the line respectively being less than or equal to 8%, strictly greater than 14%, and lying in the range 12% to 14%, by weight.

18. A line according to claim 17, the three compositions having the same multilayer interference pigment.

19. A line according to claim 1, comprising at least two, or even three or four, compositions having different respective multilayer interference pigments with the same content by weight.

20. A line according to claim 1, all of the compositions in the line being packaged in receptacles of identical shape.

21. A line according to claim 1, at least one multilayer interference pigment of at least one composition in the line including a substrate made of silica, mica, or glass.

22. A line according to claim 1, the compositions in the line being anhydrous.

23. A line according to claim 1, the compositions in the line being aqueous.

24. A line according to claim 1, the compositions in the line not being powders and the total content of multilayer interference pigment in each composition lying in the range 7% to 20%, better 5% to 15%, by weight.

25. A line according to claim 1, the compositions in the line being powders and the total content of multilayer interference pigment in each composition lying in the range 40% to 95%, better 5% to 80%, by weight.

26. A line according to claim 1, the bulk color of the compositions in the line being white.

27. A line according to claim 26, the compositions having, within their bulk, a whiteness index that is greater than or equal to 40.

28. A line according to claim 1, at least one composition in the line not including any coloring agent other than the multilayer interference pigment(s).

29. A line according to claim 1, at least one multilayer interference pigment of a composition in the line comprising at least four layers.

30. A line according to claim 1, at least one multilayer interference pigment of at least one composition in the line including a substrate made of a transparent material.

31. A line according to claim 1, the color produced by at least one multilayer interference pigment of at least one composition in the line being of dominant wavelength not lying in the range 580 to 650 nm.

32. A line according to claim 1, each composition in the line including only a single multilayer interference pigment.

33. A line according to claim 1, at least one composition in the line including at least two multilayer interference pigments.

34. A line according to claim 33, both multilayer interference pigments having layers that are made of the same material, but with at least one layer of one pigment having a thickness that is different from a corresponding layer of the other pigment.

35. A line of cosmetic compositions that are not powders, in particular that are liquid or in a stick, and that each include, in a cosmetically-acceptable medium, one or more multilayer interference pigments, with, for at least one composition, a total content of multilayer interference pigment(s) that is less than or equal to 9%, and, for one composition, a content that is greater than or equal to 11%, by weight.

36. A line according to claim 1, each composition being packaged with means that indicate the color of the composition after application to keratinous materials.

37. A line of cosmetic compositions comprising at least two compositions each comprising a cosmetically-acceptable medium and at least one multilayer interference pigment in a weight proportion greater than or equal to 7%, the multilayer interference pigments producing different colors after application, the multilayer interference pigments having a substrate preferentially made of a non opaque material for example made of mica, glass, silica, and preferentially comprising no colored coating, the proportion of diffusing pigment producing a color by a phenomenon of absorbing light being in each composition preferentially less than 0.5%.

38. A method of making up keratinous material, the method comprising applying, to keratinous materials, a composition from a line according to claim 1.
